(19) United States
(12) Patent Application Publication
(10) Pub. No.: US 2024/0076718 A1
(43) Pub. Date: Mar. 7, 2024

(54) CRISPNA FOR GENOME EDITING

(71) Applicants: UNIVERSIDAD DE GRANADA, Granada (ES); FUNDACIÓN PÚBLICA ANDALUZA PROGRESO Y SALUD, Sevilla (ES)

(72) Inventors: Francisco MARTÍN MOLINA, Sevilla (ES); Araceli AGUILAR GONZÁLEZ, Granada (ES); Noelia MALDONADO PÉREZ, Sevilla (ES); Juan José DÍAZ MOCHÓN, Granada (ES)

(21) Appl. No.: 18/260,017

(22) PCT Filed: Dec. 30, 2021
(86) PCT No.: PCT/EP2021/087887

§ 371 Date: Jun. 29, 2023

(30) Foreign Application Priority Data
Dec. 30, 2020 (ES) P202031322

(51) Int. Cl. C12Q 1/6816; C12N 9/22; C12N 15/11

(52) U.S. Cl. CPC C12Q 1/6816; C12N 9/22; C12N 15/11; C12N 2310/20; C12N 2310/3181

(57) ABSTRACT

CRISPNA, a new tool for genome editing and diagnosis. The present invention relates to methods and systems for genome editing and diagnosis, and specifically relates to use of Peptide Nucleic Acids (PNAs) to direct the Cas proteins to their DNA or RNA targets.

### FIELD OF THE INVENTION

The present invention is related to methods and systems for genome editing and diagnosis. Specifically, the invention relates to use of Peptide Nucleic Acids (PNAs) to direct the Cas proteins to their DNA or RNA targets.

### BACKGROUND OF THE INVENTION

This disclosure relates to endonuclease acid complexes, preferably Cas acid complexes and related uses thereto.

RNA-mediated adaptive immune systems in bacteria and archaea rely on Clustered Regularly Interspaced Short Palindromic Repeat (CRISPR) genomic loci and CRISPR-associated (Cas) proteins that function together to provide protection from invading viruses and plasmids. In Type II CRISPR-Cas systems, Cas9 functions as an RNA-guided endonuclease that uses a dual-guide RNA consisting of crRNA and trans-activating crRNA (tracrRNA) for target recognition and cleavage by a mechanism involving two nuclease active sites that together generate double-stranded DNA breaks (DSBs).

CRISPR utilizes an enzyme called Cas9 that uses an RNA molecule as a guide to navigate towards its targeted DNA. It then edits or modifies the DNA which can deactivate genes or insert the desired sequence to achieve a behavior. Its most promising application is genetically modifying cells to overcome genetic defects, and their potential to cure diseases like cancer.

The CRISPR/Cas9, developed in 2012 from S. pyogenes bacterial adaptive immune system, was the first genome editing (GE) tool based on CRISPR systems and also the first one that use RNA to target the nuclease to the intended site. Jinek et al (Science 337, 816-821) showed that the natural CRISPR/Cas9 can be redesigned to target any DNA sequence with the only requirement of having a PAM sequence consisting of an NGG. Although the original system uses two RNAs molecule, a crRNA sequence that is specific to the DNA/RNA target, and a tracrRNA sequence that interacts with the effector Cas9 protein, the authors showed that this system also worked using an engineered single guide RNA (sgRNA). The crRNA:tracrRNA:Cas9 or sgRNA:Cas9 complexes cause site-specific double-stranded DNA cleavage on the target sequence. This DNA damage is repair by cellular DNA repair mechanisms, principally by the non-homologous end joining (NHEJ) or by the homology-directed repair (HDR) pathways.

### CRISPR/Cas9 as a Tool for Genetic Manipulation

The first applications of the CRISPR systems used the ability of redirect Cas9 to cut into specific sites in the genome of the cells and the NHEJ or the HDR repair mechanisms as tools to introduce the desired modifications (genome editing). In the NHEJ pathway, the targeted sequences are processed by the cellular machinery generating small insertions and deletions (indels). This strategy is used to generate gene-knockout cells and organisms (transgenic animals and plants). NHEJ have also been used to recover expression of genes with frameshift mutations. When a donor DNA is delivered to the cell, together with the CRISPR system, the new DNA molecules are incorporated into the target sequence through homologous recombination, enabling precise modifications. Due to the efficacy, NHEJ-mediated GE was the first to reach the clinic. NHEJ-based GE strategies have already been used for the treatment of sickle cell disease (SCD), B-thalassemia, AIDS, and acute lymphoblastic leukemia.

The specificity of the CRISPR/Cas9 system depends on RNA-DNA recognition and it is a 20 bp crRNA (or the equivalent domain in the sgRNA), the molecule that determines the exact sequence in which the CAS9 endonuclease must bind and cut. The binding of Cas9 protein to PAM-like sequences followed by binding of the gRNA to sequences with homology to the target site generate cuts outside of the target site (off-targets). This is an important problem that is been tackle by different angles in the GE field. To start with, finding real off-targets is very challenging with no standard protocols to measure them. In fact, several studies point to higher off-targets levels to that found by standards techniques, due to the properties of the sgRNA. Reducing off-target activity of GE tools is crucial for clinical application.

Since the generation of the CRISPR/Cas9, a wide variety of new CRISPR systems have been developed based on CRISPR/Cas9 variations and also based in CRISPR systems from other bacteria. Although the composition varies between different systems, all have two modules: 1) the targeting module, based on 1 or 2 RNAs that bind the target sequence and link this sequence with the Cas protein. 2) the effector module, the Cas protein with endonuclease activity. In general, CRISPR-Cas systems fall into two classes: Class 1 systems, divided into types I, III, and IV, rely on a complex of multiple proteins to degrade foreign nucleic acids; and class 2 systems, divided into types II, V, and VI, utilize a single effector Cas protein, such as the well-studied Cas9 (a type II CRISPR system).

Most GE systems have been developed using Class 2 systems (HajizadehDastjerdi, 2019). The signature protein of Type I systems is Cas3, a single-stranded DNA nuclease and ATP-dependent helicase. Type III systems are characterized by Cas10, which assembles into a Cascade-like interference complex. Type IV systems have Csf1, a protein proposed to form part of a Cascade-like complex, though these systems consist of isolated cas genes without an associated CRISPR array. Type V systems also contain a Cas9-like single nuclease, either Cpf1(Cas12a), C2c1, or C2c3. Type VI systems have Cas13 (formerly C2c2), a large protein with two HEPN (higher eukaryotes and prokaryotes nucleotide-binding) RNase domains (Shmakov et al., 2015).

The types based on multi-subunit effector complexes (class 1 systems) have also been used, although their architecture makes them less manageable, for gene editing. However, its large size and stable binding make them more appropriate for other applications, such as transcription silencing (Rath et al, 2015).

There are certain problems with CRISPR such as unwanted off-target mutations. It works by cutting the double-stranded DNA at precise locations in the genome. When the cell's natural repair process takes over, it can cause damage. Further, it could create unwanted off-target mutations where the modified DNA is inserted at the cut site.

US20190032036A1 describes a modification of the CRISPR enzyme to solve the off-target effect. Because DNA is negatively charged, it binds itself to a groove in Cas9 protein which is positively charged. Inventors replaced some of the positively charged amino acids with neutral ones to decrease the binding of “off-target” sequences.

Zhang's team found that mutations in three amino acids dramatically reduced “off-target” cuts. They have created a newly engineered enzyme—“enhanced” S. pyogenes Cas9, or eSpCas9, which will be useful for specific genome editing applications.

Other solution is the implementation of second guide RNA (US20170247671A1). During a CRISPR based edit, a strand of molecules called a guide RNA leads the DNA-slicing protein Cas9 to the section of DNA targeted for editing. Once the guide RNA binds to the DNA, Cas9 makes the cut so that new DNA can be inserted or deleted, and an additional second guide RNA can be used having a blocking guide sequence that is complementary to an off-target nucleic acid sequence. There could be quite a bit of randomness in what happens during these CRISPR edits, and that randomness can potentially create unexpected outcomes. To minimize them, in U.S. Pat. No. 10,354,746B they developed an algorithm that takes in data and identifies cleavage locations of Cas9 nuclease and selects the nuclease having fewest off-target cleavage locations.

To validate off-target sites, an in vitro Cas9-digested whole-genome sequencing technique, Digenome-seq, was developed in US20190153530A1. This in vitro method yields sequences that can be computationally identified to profile genome-wide Cas9 off-target effects in human cells. Digenome-seq is a robust, sensitive, unbiased, and cost-effective method for profiling genome-wide off-target effects of programmable nucleases including Cas9.

In Anzalone et al (Nature 576, 149-157, 2019) they developed a technique (CRISPR prime) that has the potential to fix more than 90% of known genetic diseases including Tay-Sachs disease. This versatile and precise genome editing method works by directly writing new genetic information into a specified DNA site using a catalytically impaired Cas9 endonuclease fused to an engineered reverse transcriptase, programmed with a prime editing guide RNA (pegRNA) that both specify the target site and encodes the desired edit. CRISPR prime also offers the versatile function of multi-letter base-editing [Can tackle four DNA letters mutation genetic disorders like Tay-Sachs] with minimized DNA damage, less off-target effect, and efficient DNA healing. A guide RNA called pegRNA guides the Cas9 enzyme to snip out only a single strand of DNA and prevents the double-strand breaks which can induce unintended disruptions. After that, the reverse transcriptase enzyme directly copies the edited genetic information contained in the pegRNA to the targeted genomic site. This helps to generate cells, which will help patients to recover and heal as well as help develop new vaccines against deadly diseases.

CRISPR/Cas systems are powerful technologies that are changing the way scientists tackle unsolved problems in basic biology, therapy and diagnosis. In its present forms, the different CRISPR/Cas systems require RNA molecules (crRNAs or sgRNAs) to direct the different Cas proteins to their DNA or RNA targets. In spite of their potency and specificity, the RNA molecules are unstable and can allow several mismatches when binding to their target, and this can lead to lack of reproducibility, mutations in undesired genes and false positives.

### DESCRIPTION OF THE FIGURES

FIGS. 1 and 2. Structure of a typical PNA molecule (FIG. 1) and PNA paired with DNA (FIG. 2). Typically, the total number of PNA units (a+b+c) in the oligomer may be in the range of 5-50, typically 18-30 G are selected from either a hydrogen or an organic moiety such as polyethylene glycol. P is either an H or organic moieties such as biotin, acrylamide, acetate, fluorophores, alkynes, azides, maleimaides, thiols, digoxigenin, disulfur. NB is a nucleobase; and b≥0.

FIG. 2. CRISPNA design for type II CRISPR systems. The structures of the CRISPR-II system (Left) and CRISPNA-II (Right) are shown. In CRISPR, the Cas9 is guided by an RNA molecule, the crRNA, that binds to the target sequence and to the tracrRNA-Cas9. The CRISPNA design replaces the crRNA by the crPNA that will also bind the target sequence and the tracrRNA-Cas9. The final design will have three components: 1—Cas9, 2—a variable crPNA that binds the chosen target sequence and 3—a constant tracrRNA that will bind Cas9 and the crPNA.

FIG. 3. CRISPNA design for type II CRISPR systems. The structures of the CRISPR-II system (Left) and CRISPNA-II (Right) are shown. In CRISPR, the Cas9 is guided by an RNA molecule, the crRNA, that binds to the target sequence and to the tracrRNA-Cas9. The CRISPNA design replaces the crRNA by the crPNA that will also bind the target sequence and the tracrRNA-Cas9. The final design will have three components: 1—Cas9, 2—a variable crPNA that bind the chosen target sequence and 3—a constant TracrRNA that will bind Cas9 and the crPNA.

FIG. 4. Dual CRISPNA design for Type V/VI CRISPR systems. The structures of the CRISPR-V/VI systems (Left) and CRISPNA-V/VI (Right) are shown. In CRISPR, the Cas is guided by a sgRNA molecule, the crRNA, that binds to the target sequence and to the Cas. To develop a CRISPNA design from these systems, we need first to dissociate the functions of DNA binding (crPNA) and Cas binding (tracrRNA) in these systems. The final design will have three components: 1—Cas, 2—a variable crPNA that binds the chosen target sequence and 3—a constant tracrRNA-typeV/VI that will bind Cas and the crPNA.

FIG. 5. Single CRISPNA design for Type V/VI CRISPR systems. The structures of the CRISPR-V/VI systems (Left) and CRISPNA-V/VI (Right) are shown. In CRISPR, the Cas is guided by sgRNA molecule, the crRNA, that binds to the target sequence and to the Cas. To develop a single CRISPNA design from this system, we constructed a chimeric RNA-PNA molecule. The RNA (in green) will bind the Cas protein and the PNA domain will bind the target sequence. The final design will have two components: 1—Cas, 2—a RNA-PNA chimera that bind the chosen target and the Cas nuclease.

FIG. 6. Strategy to analyze efficiency and specificity of CRISPNA compared to CRISPR for genome editing. The different RNPs harboring CRISPR (left) or CRISPNA (Right) will be nucleofected in Target cells. 4-6 days later we will analyze the cutting efficacy by PCR, sequencing and TIDE analysis and the specificity by measuring off-targets. Due to the PNAs properties, the CRISPNAs should be able to recognize their target sequences in a more specific and stable manner compared to CRISPR leading to enhanced efficacy and less off targets. Different crPNAs configurations will be analyzed in the search for the best PNA design to be adapted to CRISPNAs systems.

FIG. 7. A) CRISPNA design for Cas13 applications A) The crPNAs were designed incorporating a RNA target sequence specific for SARS-Cov-2 and a ΔtracrRNA13-binding domain that will bridge the target sequence with the Cas13. The newly designed ΔtracrRNA13 RNA (bottom) lacks the target sequence domain (now provided by the PNA) but incorporates a PNA binding domain and a Cas13 binding domain. B) Detection of Cas/crPNA/ΔtracrRNA13 (CRISPNA13-Cov) complex. Cas13 were incubated with control crRNA13 (Right panel) or with crPNA+ΔtracrRNA13 (left panel), the samples were run on a native polyacrylamide gel and stained with Gel-Red for nucleic acid staining. A ribonucleoprotein (large band containing Cas13 and RNA) was formed with both, the control CRISPR/Cas13 (line 10, right panel) as well as with CRISPNA13_Cov2 (line 5, left panel). The numbers corresponded to: 1. crPNA; 2. ΔtracrRNA13; 3. Cas13; 4. crPNA+ΔtracrRNA13; 5. crPNA+ΔtracrRNA13+Cas13 (Cas13 complex); 6. crRNA13 (mimicking crPNA13); 7. ΔtracrRNA13; 8. Cas13; 9. crRNA13+ΔtracrRNA13; 10. crRNA13+ΔtracrRNA13 (Cas13 RNP dual).

FIG. 8. CRISPNA efficiently edited the genome of eukaryotic cells. A) Chromatograms of K562 SEWAS84S-C1 cells (harbouring 1 copy of eGFP) non edited (top) versus edited with CRISPNA (bottom) for eGFP sequence. B) The efficiency of genome editing of the control sample versus the edited one with CRISPNA by TIDE analysis. C) % of indel distribution in the CRIPNA edited sample obtained by TIDE analysis. Of note, a control electroporating PNAs without Cas9 is lacking to be completely sure that CRISPNA require Cas9 to edit the genome of these cells. Indeed, although the design of these PNAs lack a triplex helix configuration, we cannot completely eliminate the possibility that the edition observed is due to PNA binding to its target.

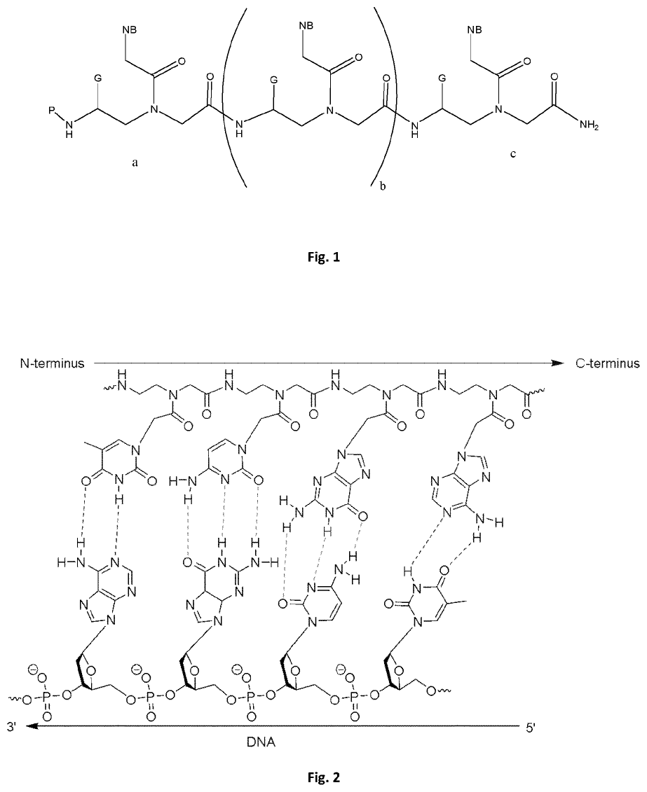

### DESCRIPTION OF THE INVENTION

This invention combines the versatility of CRISPR-associated enzymes (Cas) with the robustness, stability and specificity of peptide nucleic acids (PNAs) to generate the “CRISPNA” technology, with improved characteristics over CRISPR systems.

Peptide Nucleic Acids (PNAs) are artificially synthetic oligonucleotides that display higher affinity to complementary DNA and RNA than do normal oligonucleotides. Therefore, PNA-RNA and PNA-DNA bindings are more stable and specific than RNA-DNA. In addition, their uncharged backbone makes PNAs extremely stable in biological fluids, since they are resistant to proteases and nucleases.

CRISPNA is an alternative to CRISPR systems and can be used to improve the efficacy and specificity of all applications in which CRISPR has been used. Therefore, CRISPNA can be used to manipulate DNA and RNA, as well as a tool to detect and/or image specific DNA and RNA sequences. Importantly, it can be used in living cells as well as in different fluids.

The present invention uses PNAs, instead of crRNAs or sgRNAs, to direct the Cas proteins to their DNA or RNA targets.

Thus, a first aspect of the invention relates to the use of PNAs to direct the Cas proteins to their DNA or RNA targets.

“Peptide Nucleic Acids” or “PNAs” are synthetic mimics of oligonucleotides in which the sugar-phosphate backbone is replaced by a peptide to which the nucleobases are linked.
They display higher affinity to complementary DNA and RNA than do normal oligonucleotides. Therefore, PNA-RNA and PNA-DNA binding are more stable than RNA-DNA.
Compared to RNA, PNAs are also more specific in respect to their targets due to their rigid conformation.
Their uncharged backbone makes PNAs extremely stable in biological fluids since they are resistant to proteases and nucleases.
Gamma modifications in their backbone enhances DNA invasion without the need of homopurine stretches.

### Composition and Kit of Parts of the Invention (System of the Invention)

A second aspect of the invention relates to a CRISPNA complex or system (for recognition and cleavage of a target nucleotide, preferably a target DNA, sequence) comprising:
(i) optionally, a Cas polypeptide or a polynucleotide encoding a Cas polypeptide; and
(ii) a guide system comprising:
   a) a scaffold RNA (tracrRNA) bound, binding or capable of binding the/a Cas polypeptide, or a polynucleotide encoding said tracrRNA, and
   b) a guide PNA (crPNA) bound, binding or capable of binding the tracrRNA of a) and capable of binding the target sequence which should be recognized and cleaved.

Preferably, said complex or system forms or is comprised in a composition or in a kit of parts, hereinafter composition or kit of parts of the invention.

In a preferred embodiment, the guide system is a single guide system comprising an RNA-PNA chimera (crRPNA) in which the RNA domain will bind the Cas polypeptide and the PNA domain will bind the target oligonucleotide sequence.

In the present specification, “Cas polypeptide” refers to an endonuclease, preferably a CRISPR endonuclease. Cas polypeptides (part of the CRISPR or CRISPNA system) are endonucleases, meaning that they cut DNA somewhere in the middle of a strand, rather than taking bases off the end. Cas enzymes are guided to its cut a site by an single guide RNA (sgRNA) which uniquely targets the DNA sequence to which it is complementary thereto. This means that instead of engineering a whole new protein, if we want to target a specific site we can simply change the sgRNA sequence. The Cas polypeptide in order to start the cleaving reaction needs a double interaction with the DNA and the tracrRNA. Most Cas proteins, also, in order for the reaction to take place—need a consensus sequence named PAM. Each PAM is specific for each Cas polypeptide.

The “tracrRNA” or “trans-activating crRNA” is made of up of a longer stretch of bases that are constant and provide the “stem loop” structure bound by the CRISPR or CRISPNA nuclease (i.e. Cas9).

When tracrRNA hybridizes with the crPNA they form a guide RNA-PNA which “programmably” targets CRISPR or CRISPNA nucleases to DNA or RNA sequences depending on the complementarity of the crPNA and the presence of other DNA or RNA features (PAM sequences recognized by the different Cas nucleases).

The “single guide RNA” or “sgRNA” is a single RNA molecule that contains both the custom-designed short crRNA sequence fused to the scaffold tracrRNA sequence. sgRNA can be synthetically generated or made in vitro or in vivo from a DNA template.

In the context of the present invention, the “guide system” of the invention comprising the tracrRNA sequence and the crPNA, is equivalent to the sgRNA. As said previously, the guide system may be also a RNA-PNA chimera, named crRPNA, that is equivalent to the sgRNA.

The “kit of parts” or “system” of the invention refers to a combination of a set of components suitable for targeting specific DNA or RNA sequences which may or may not be administered together. The components of the kit (the CRISPR enzyme—Cas polynucleotide—and the guide system) can be provided in separate vials (in the form of “kit of parts”) or in a single vial.

Parts of the kit of the invention can be jointly or separately sold/administered.

It should be emphasized that the term “kit of parts” in this specification, means that the components of the system of the invention (CRISPR or CRISPNA enzyme/Cas polynucleotide, tracRNA, crPNA—or the RNA-PNA chimera—) do not need to be present in the same composition, in order to be available for their combined, separate or sequential application. Thus, the expression “kit of parts” implies that a true combination does not necessarily result, in view of the physical separation of the components.

In some embodiments, the CRISPNA complex or system comprises one or more nuclear localization sequences of sufficient strength to drive accumulation of said CRISPNA complex into the nucleus of eukaryotic cells. Then, in a preferred embodiment, the CRISPNA complex or system, the composition, or the kit of parts of the invention comprises nuclear localization sequences.

In a preferred embodiment, the guide system from the CRISPNA complex or system, the composition, or the kit of parts of the invention comprises or consists of a structure of Formula (I):

Ac—NH—Y-link-Z—CONH₂ Formula (I)

wherein
Y: represents the PNA guide domain, preferably a sequence of 5-35 nucleobases that hybridizes the target sequence.
Link: represents a 1-15, more preferably 1-10, and still more preferably 1-7 aminoethyl glycine, or aminoethyl glycine derivative or analogue, linker between the tracRNA-binding domain and the domain that binds the target sequence.
Z: is a PNA sequence binding the tracrRNA and comprising or consisting of more than 5 nucleobases, more preferably 6-14 nucleobases and still more preferably about 10 nucleobases.

In another preferred embodiment, the guide system from the CRISPNA complex or system, the composition, or the kit of parts of the invention is a RNA-PNA chimera (crRPNA), comprising or consisting of a structure of Formula (II):

Ac—NH—Y-link-RNA Formula (II)

wherein
Y: represents the PNA guide domain, preferably a sequence of 5-35 nucleobases that hybridizes the target sequence.
Link: represents a 1-15, more preferably 1-10, and still more preferably 1-7 aminoethyl glycine or nucleotide (of RNA) linker between the RNA and the domain that binds the target sequence.
RNA: represents the tracRNA

In another preferred embodiment, the Cas polypeptide or the polynucleotide encoding the same is selected from the group consisting of: Cas1, Cas2, Cas3, Cas4, Cas5, Cas6, Cas7, Cas8, Cas9, Cas10, Cas11, Cas12, Cas13, Csy1, Csy2, Csy3, Cse1, Cse2, Csa5, Csn2, Csm2, Csm3, Csm4, Csm5 and/or Csm6

In another preferred embodiment, the Cas polypeptide belongs to the type II, type V or type VI CRISPR or CRISPNA systems.

Another embodiment refers to the CRISPNA complex or system, the composition, or the kit of parts of the invention, wherein the Cas polypeptide is Cas9 and the guide system comprises:
a) a tracrRNA from any CRISPR/Cas9 system, as the one described in [CRISPR-Cas9 Structures and Mechanisms. Jiang F, Doudna J A. Annu Rev Biophys. 2017 May 22;46:505-529. doi: 10.1146/annurev-biophys-062215-010822. Epub 2017 Mar. 30]; and
b) a guide PNA (crPNA) binding the tracrRNA of a) and the target sequence next to a PAM sequence (NGG)

More preferably the guide system comprises:
(I) A tracrRNA(9) comprising, consisting essentially of or consisting of SEQ ID NO: 1:

SEQ ID NO 1
5′AGCAUAGCAAGUUAAAAUAAGGCUAGUCCGUUAUCAA
CUUGAAAAAGUGGCACCGAGUCGGUGCUUU 3′.

and
(II) A guide PNA (crPNA) of formula I, wherein Z comprises or consists of SEQ ID NO: 2

SEQ ID NO 2
guuuaaggcuaugcu.

In another preferred embodiment, the guide PNA (crPNA) of the CRISPNA complex or system, the composition, or the kit of parts of the invention has the structure of a RNA-PNA chimera (crRPNA), having the structure previously defined.

Double-stranded DNA (dsDNA) recognition and cleavage by Cas9 strictly require the presence of a “PAM sequence” or “protospacer-adjacent-motif” (for Cas9 recognition, nGG, n=any nucleotide)in the non-complementary, DNA strand (ntDNA) and the complementarity of the target DNA strand (tDNA) to the 10-12 nucleotide (nt) PAM-proximal “seed” region in the guide RNA. Once the guide RNA binds to the target sequence, the Cas9 enzyme recognizes the site and makes a double strand break in the DNA sequence 3-4 nucleotides upstream the PAM sequence. In nature, Cas nucleases derived from different bacterial species recognize different PAMs. In the case of the spCas9 (Streptococcus pyogenes) the PAM sequence is NGG. So the guide system (formed by the tracrRNA and the crPNA—instead the sgRNA—) targets the Cas9 where you want it to cleave and the interaction with the PAM is needed for the conformational rearrangements of the Cas9 to start cleaving the DNA.

Another preferred embodiment refers to the CRISPNA complex or system, the composition, or the kit of parts of the invention, wherein the Cas polypeptide is selected from the list consisting of Cas 5, Cas 7, Cas 12 and/or Cas 13; and the guide system comprises:
(I) A 25-60 nt tracrRNA derived from the Cas 5, Cas 7, Cas 12 and/or Cas 13crRNA respectively, that:
   Maintains the first 18-22 nts from the 5′ for binding Cas 5, Cas 7, Cas 12 and/or Cas 13 protein;
   Lack the last 15-25 nts from the 3′ (guide domain); and
   Include a 5-15 nts sequence for binding the guide PNA;
(II) A guide PNA (crPNA) binding the respective tracrRNA and the target sequence.

Another preferred embodiment refers to the CRISPNA complex or system, the composition, or the kit of parts of the invention, wherein
when the Cas is Cas12, the tracrRNA preferably comprises or consists of a structure of Formula (III):

5′ SEQ ID NO: 3-Z′ 3′
Formula (III)
(5′ UAAUUUCUACUCUUGUAGAU-Z′ 3′)
SEQ ID NO: 3:
UAAUUUCUACUCUUGUAGAU

when the Cas is Cas13, the tracrRNA preferably comprises or consists of a structure of Formula (IV):

5′SEQ ID NO: 5-Z′3′
Formula (IV)
(5′ gauuuagaaccccaaaaacgaaggggacuaaaac-Z′ 3′)

and wherein Z′ is a polynucleotide having between 5 and 70 nucleotides that hybridizes the Z sequence of the PNA sequence binding the tracrRNA of formula I.

### Non-Viral Vectors

Other aspect of the present invention refers to a non-viral vector, hereinafter non-viral vector of the invention, comprising the system, the composition or the kit of parts of the invention.

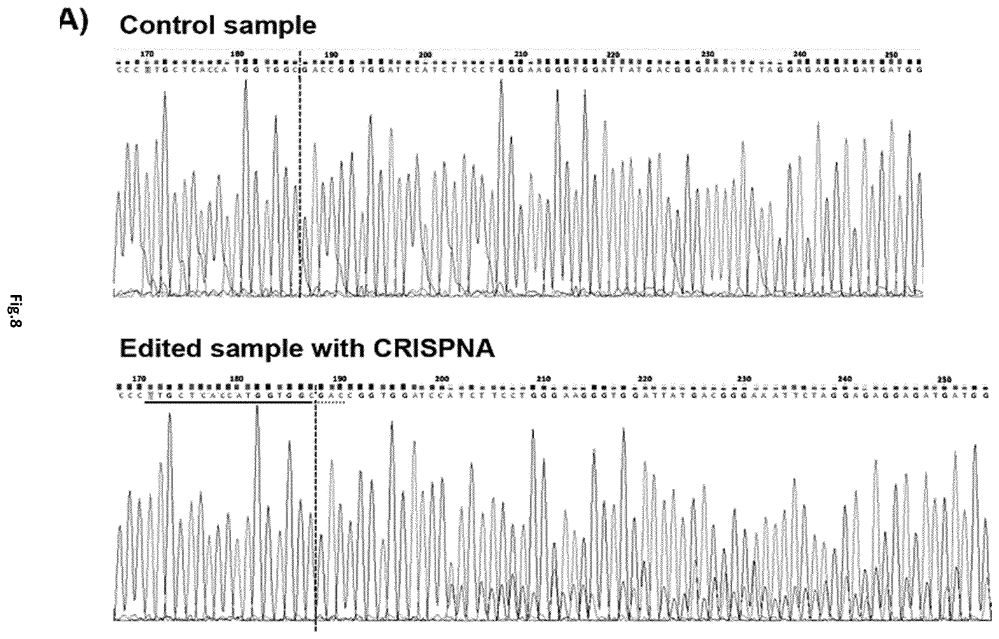

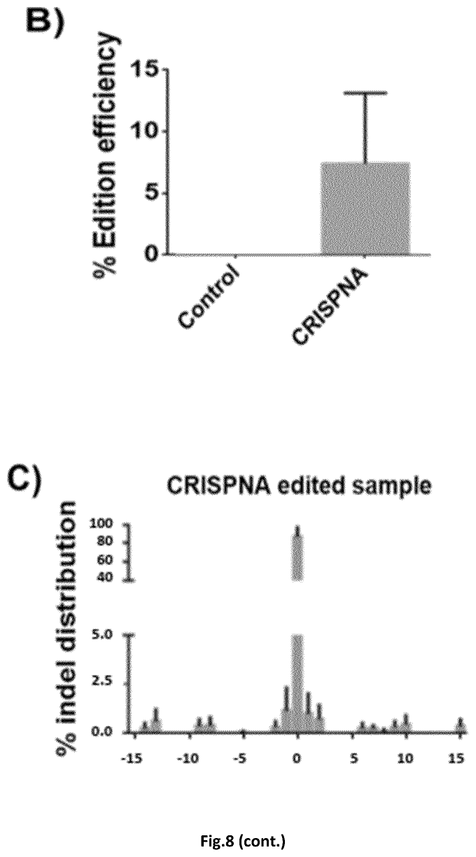

The Cas polypeptide, the tracrRNA and the crPNA can form a ribonucleopeptide complex than can act as non-viral vectors (FIG. 6)

In one embodiment of the present aspect the non-viral vector of the invention is transferred into a target cell using a non-viral system. More preferably the non-viral system is selected from the list consisting on: an electroporator, a liposome, a polycation, a nanoparticle, or combinations thereof.

### Uses of the Composition, Kit of Parts and Non-Viral Vectors of the Invention

Another aspect of the invention refers to the use of the CRISPNA complex or system, the composition, the kit of parts or the non-viral vectors of the invention for genome editing. The present invention can be used, without limitation, to generate gene-modified cells and organisms (transgenic animals and plants). Also, can be used to modulate gene expression of cells and organisms. For example, for increasing expression of a chromosomal sequence in a cell or embryo.

Preferably, the cell is a human cell, a non-human mammalian cell, a stem cell, a non-mammalian vertebrate cell, an invertebrate cell, a plant cell, or a single cell eukaryotic organism.

Then, another aspect of the invention refers to the CRISPNA complex or system, the composition, the kit of parts or the non-viral vectors of the invention for use in medicine.

Another aspect of the invention refers to the CRISPNA complex or system, the composition, the kit of parts or the non-viral vectors of the invention for the prevention, amelioration or treatment of a disease or disorder.

The CRISPNA-based gene editing can be used to inactivate or correct gene mutations causing diseases, for example dystrophies and/or microsatellite expansion diseases, thereby providing a gene therapy approach for these groups of diseases.

Then, another aspect refers to the CRISPNA complex or system, the composition, the kit of parts or the non-viral vectors of the invention for the treatment of genetic diseases.

The guided nuclease system of the present invention can target any specific region of the microorganism's genome (bacteria, virus . . . ). Thus, the CRISPNA complex or system, the composition, the kit of parts or the non-viral vectors of the invention can be plausibly used for killing a bacterium contacting the bacterium and creating a double-stranded break in the chromosomal DNA of the bacterium. Another strategy could be making a bacterium more susceptible to an antibiotic, cleaving an antibiotic resistance gene encoded by the bacterium. Also, methods of the invention may be used to remove latent virus genetic material from a host organism, without interfering with the integrity of the host's genetic material.

Then, another aspect refers to the CRISPNA complex or system, the composition, the kit of parts or the non-viral vectors of the invention for the treatment of infectious diseases.

The CRISPNA complex or system, the composition, the kit of parts or the non-viral vectors of the invention could be used to therapeutically target oncogene mutations or to repair defective tumor suppressor genes. That is, they can be used to inactivate or correct oncogene mutations causing cancer, thereby providing a gene therapy approach for treating the underlying causes of cancer.

Also, the CRISPNA complex or system, the composition, the kit of parts or the non-viral vectors of the invention can be plausibly used to eliminate immune checkpoint genes from T cells for cancer immunotherapy approaches.

Also, the CRISPNA complex or system, the composition, the kit of parts or the non-viral vectors of the invention could be used to generated universal CAR-T cells by eliminating the TCR.

Then, another aspect of the invention relates to the CRISPNA complex or system, the composition, the kit of parts or the non-viral vectors of the invention for preventing, inhibiting, or treating cancer in a subject. For example, the guide system comprises at least one targeted genomic sequence, such as an oncogenic mutation or tumor suppressor gene.

Another aspect of the invention refers to a method, hereinafter first method of the invention, for generating specific cleavage in a double stranded DNA, in a single stranded DNA or in a single stranded RNA using the CRISPNA complex, the composition, the kit of parts or the non-viral vectors of the invention. More preferably said cleavage is in a cell in vitro or ex vivo. Still more preferably said cleavage is in a cell in vivo and the delivery of the ribonucleo-peptide complex is performed as mentioned previously.

In another preferred embodiment, the aim of such cleavage is to modify the target sequence. In another preferred embodiment, the aim of such cleavage is to repair existing mutations. In another preferred embodiment, the aim of such cleavage is to disrupt the function of a functional protein. In another preferred embodiment, the aim of such cleavage is to restore the function of a mutated protein.

Another aspect of the invention refers to a method. Hereinafter second method of the invention, for specific binding to double stranded DNA, a single stranded DNA or a single stranded RNA, using the CRISPNA complex, the composition, the kit of parts or the non-viral vectors of the invention wherein the Cas polypeptides are mutated for their cleavage activity. More preferably said binding is in a cell in vitro or ex vivo and the delivery of the ribonucleopeptide complex is performed as mentioned previously.

In another preferred embodiment, the aim of such binding is to visualize the target sequence.

In another preferred embodiment, the aim of such binding is to detect the target sequence.

Another aspect of the invention refers to the composition, the kit of parts or the non-viral vectors of the invention for monitoring a disease or disorder.

### Diagnostic Uses of the Invention

Some traditional diagnostic methods such as PCR are time consuming assays that require multiple steps and specific equipment. So, an ideal rapid diagnostic test would be sensitive and specific, easy to perform and affordable. CRISPR-based assays require minimal to no equipment and can be run as single reaction tests. They are easy to use and can deliver fast and accurate results. The CRISPNA system could also take these advantages but increasing efficacy and specificity of the systems.

Several groups have taken advantage of the characteristics of class 2 Type V and Type IV CRISPR systems, based mainly on Cas12a and Cas13a nucleases, respectively, developing different platforms for rapid and accurate nucleic acids detection. Firstly, the Professor Doudna and her team demonstrated that combining the processing and interference activities of Cas13a it is possible the detection of cellular transcripts (East-Seletsky, et al. (2016). Nature 538, 270-273).

Later, the RNA-activated ssRNA-degradation activity of Cas13a was functionalized to create the SHERLOCK (Specific High Sensitivity Enzymatic Reporter UnLOCKing) platform. A paper-based assay that uses isothermal amplification and Cas13a for in vitro detection of specific strains of Dengue and Zika virus with attomolar sensitivity, distinguishes pathogenic bacteria, identifies low frequency DNA mutations (e.g. SNP) correlated with cancer and other diseases, or human genotyping⁷. Combination of orthogonal CRISPR enzymes, which present discrete crRNA and substrate, and other advances, promoted a more advanced SHERLOCK platform, known as SHERLOCKv2, which allows simultaneously detection of Zika and Dengue virus, and mutations in liquid biopsy samples⁸. Similarly, exploiting the multiple-turnover nuclease activity of Cas12a Chen et al have developed DETECTR (DNA endonuclease-targeted CRISPR trans reporter), a method that with attomolar sensitivity allows specific nucleic acid detection between two types of human papillomavirus (HPV). This platform has been applied for detection of betacoronavirus severe acute respiratory syndrome (SARS)-CoV-2 (COVID-19) from respiratory swab RNA extracts in a portable, easy to perform, rapid and accurate manner.

CRISPR-Cas9 system has also been used for developing infectious disease diagnostics. In fact, it can be also applied to fight against the antibiotic-resistance bacteria problem, targeting virulence and resistance genes. It has also been used in the field of cancer genomics. For instance, to enrich KRAS mutations so that depleting wild-type copies to increase the downstream sensibility of the assay.

CRISPR/Cas 9 is also a very powerful tool to do targeted sequencing of long DNA fragments without the need of amplifying DNA. This is key to do methylation studies of long DNA regions¹²

Then, another aspect of the invention refers to the first or the second method of the invention for the diagnosis of a disease or disorder.

### Other Definitions

Unless otherwise specified, “a,” “an,” “the,” and “at least one” are used interchangeably and mean one or more than one.—Amino acid residues in a polypeptide sequence are designated herein according to the one-letter code, in which, for example, Qmeans Glutamine residue, R means Arginine residue and D means Aspartic acid residue.
Amino acid substitution means the replacement of one amino acid residue with another, for instance the replacement of an Arginine residue with a Glutamine residue in a peptide sequence is an amino acid substitution.—Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
“As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally occurring nucleotides (such as DNA and RNA), or analogues of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.
The terms “vector” or “vectors” refer to system capable of transporting the desire molecule, or combination of molecules into the target cell. A “vector” in the present invention includes, but is not limited to, a viral vector, and a non-viral vector such as plasmid, a linear RNA or DNA, and ribonucleoprotein (RNP) complexes. RNA and DNA may consists of a chromosomal, non-chromosomal, semi-synthetic or synthetic nucleic acids. Large numbers of suitable vectors are known to those of skill in the art and commercially available.
Delivery vectors and vectors can be associated or combined with any cellular permeabilization techniques such as sonoporation or electroporation or derivatives of these techniques.
by “mutation” is intended the substitution, deletion, insertion of up to one, two, three, four, five, six, seven, eight, nine, ten, eleven, twelve, thirteen, fourteen, fifteen, twenty, twenty five, thirty, forty, fifty, or more nucleotides/amino acids in a polynucleotide (cDNA, gene) or a polypeptide sequence. The mutation can affect the coding sequence of a gene or its regulatory sequence. It may also affect the structure of the genomic sequence or the structure/stability of the encoded mRNA.
by “variant(s)”, it is intended a repeat variant, a variant, a DNA binding variant, a TALE-nuclease variant, a polypeptide variant obtained by mutation or replacement of at least one residue in the amino acid sequence of the parent molecule.
by “functional variant” is intended a catalytically active mutant of a protein or a protein domain; such mutant may have the same activity compared to its parent protein or protein domain or additional properties, or higher or lower activity.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.
“similarity” describes the relationship between the amino acid sequences of two or more polypeptides. BLASTP may also be used to identify an amino acid sequence having at least 70%, 75%, 80%, 85%, 87.5%, 90%, 92.5%, 95%, 97.5%, 98%, 99% sequence similarity to a reference amino acid sequence using a similarity matrix such as BLOSUM45, BLOSUM62 or BLOSUM80. Unless otherwise indicated a similarity score will be based on use of BLOSUM62. When BLASTP is used, the percent similarity is based on the BLASTP positives score and the percent sequence identity is based on the BLASTP identities score. BLASTP “Identities” show the number and fraction of total residues in the high scoring sequence pairs which are identical; and BLASTP “Positives” show the number and fraction of residues for which the alignment scores have positive values, and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means. A polynucleotide encoding such a functional variant would be produced by reverse translating its amino acid sequence using the genetic code.

The term “subject” or “patient” as used herein includes all members of the animal kingdom including non-human primates and humans.

The above written description of the invention provides a manner and process of making and using it such that any person skilled in this art is enabled to make and use the same, this enablement being provided in particular for the subject matter of the appended claims, which make up a part of the original description.

Where a numerical limit or range is stated herein, the endpoints are included. Also, all values and subranges within a numerical limit or range are specifically included as if explicitly written out.

The above description is presented to enable a person skilled in the art to make and use the invention and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown but is to be accorded the widest scope consistent with the principles and features disclosed herein.

It is noted herein that this invention is also directed to the following clauses or embodiments
1.—The use of PNAs to direct the Cas proteins to their DNA or RNA targets.
2.—The use of PNA-RNA chimeric molecules to direct the Cas proteins to their DNA or RNA targets.
3.—A composition or a kit of parts comprising:
   (i) a Cas polypeptide or a polynucleotide encoding a Cas polypeptide;
   (ii) a guide system comprising:
      a) An scaffold RNA (tracrRNA) binding the Cas protein and the guide PNA, or a or a polynucleotide encoding said tracrRNA,
      b) A guide PNA (crPNA) binding the tracrRNA and the target sequence
4.—The composition or the kit of parts according to clause 3, wherein the guide PNA (crPNA) from the composition or the kit of parts of the invention has the structure of Formula (I):

Ac—NH—Y-link-Z—CONH₂ Formula (I)
and wherein
   Y: represents the PNA guide domain, a sequence of 5-35 nucleobases that hybridizes the target sequence.
   Link: represent a 1-15, more preferably 1-10, and still more preferably 1-7 aminoethyl glycine, or aminoethyl glycine analogous, linker between the tracRNA-binding domain and the domain that binds the target sequence.
   Z: is a PNA sequence binding the tracrRNA and having more than 5 nucleobases, more preferably 6-14 nucleobases and still more preferably about 10 nucleobases.
5.—The composition or the kit of parts according to clause 3, wherein the guide PNA (crPNA) has the structure of a RNA-PNA chimera (crRPNA), having
the structure of Formula (II):

Ac—NH—Y-link-RNA Formula (II)
Wherein
   Y: represents the PNA guide domain, a sequence of 5-35 nucleobases that hybridizes the target sequence.
   Link: represent a 1-15, more preferably 1-10, and still more preferably 1-7 aminoethyl glycine or nucleotides (of RNA) linker between the RNA and the domain that binds the target sequence.
   RNA: represents the tracRNA
6.—The composition or the kit of parts according to clause 2, wherein the guide PNA (crPNA) have the structure of Formula (I):

NH₂—Y-link-Z—CONH₂ Formula (I)
Wherein
   Y: represents the PNA guide domain, a sequence that hybridizes the target sequence.
   Link: represent a 1-7 aminoethyl glycine linker between the tracRNA-binding domain and the domain that binds the target sequence.
   Z: is a sequence binding tracrRNA.
7.—The composition or the kit of parts according to any one of clauses 2-6, wherein the Cas polypeptide belong to the type II, type V or type VI CRISPR systems, and preferably is selected from the group consisting on: Cas1, Cas2, Cas3, Cas4, Cas5, Cas6, Cas7, Cas8, Cas9, Cas10, Cas11, Cas12, Cas13, Csy1, Csy2, Csy3, Cse1, Cse2, Csa5, Csn2, Csm2, Csm3, Csm4, Csm5 and/or Csm6.
8.—The composition or the kit of parts according to any one of clauses 2-7, wherein the Cas polypeptide is Cas9 and the guide system comprises:
   a) A tracrRNA from any CRISPR/Cas9 system.
   b) A guide PNA (crPNA) binding the tracrRNA and the target sequence next to a PAM sequence (NGG)
9.—The composition or the kit of parts according to clause 8, wherein the guide system comprises:
   (I) A tracrRNA(9) containing the SEQ ID NO: 1

5′ AGCAUAGCAAGUUAAAAUAAGGCUAGU
CCGUUAUCAACUUGAAAAAGUGGCACCGAG
UCGGUGCUUU 3′

(II) A guide PNA (crPNA) according to clause 3, wherein Z is SEQ ID NO: 2

guuuaaggcuaugcu

10.—The composition or the kit of parts according to any one of clauses 2-9, wherein the Cas polypeptide is selected from the list consisting on: Cas 5, Cas 7, Cas 12 and/or Cas 13; and the guide system comprises:
   (I) a 25-60 nt tracrRNA derived from the Cas 5, Cas 7, Cas 12 and/or Cas 13crRNA respectively, that:
      Maintains the first 18-22 nts from the 5′ for binding Cas 5, Cas 7, Cas 12 and/or Cas 13 protein
      Lack the last 15-25 nts from the 3′ (guide domain).
      Include a 6-15 nt sequence for binding the guide PNA
   (II) A guide PNA (crPNA) binding the respective tracrRNA and the target sequence.
11.—The composition or the kit of parts according to claim 10, wherein
when the Cas polynucleotide is Cas 12, the tracrRNA has the structure of Formula (II)

Formula (III)
5′ UAAUUUCUACUCUUGUAGAU-Z′ 3′

when the Cas is Cas13, the tracrRNA has the structure of Formula (IV)

Formula (IV)
5′ gauuuagaaccccaaaaacgaaggggacuaaaac-Z′ 3′

and wherein Z′ is a polynucleotide having between 5 and 70 nucleotides that hybridizes the Z sequence of the PNA guide as described in clause 3.
12.—A non-viral vector comprising the composition or the kit of parts according to any one of clauses 2-11, wherein the Caspolipetide, the tracrRNA and the crPNA are mixed forming a ribonucleo-peptide complex.
13.—The non-viral vector according to clause 12, that is transferred into a target cell using a non-viral system.
14.—The non-viral vector according to clause 13, wherein the non-viral system is selected from the list consisting on: an electroporator, a liposome, a polycation, a nanoparticle, or combinations thereof.
15.—The composition or the kit of parts according to any one of clauses 2-11, or the non-viral vector according to any one of clauses 12-14, for use in medicine.
16.—The composition or the kit of parts according to any one of clauses 2-11, or the non-viral vector according to any one of clauses 12-15, for the prevention, amelioration, treatment or monitoring of a disease or disorder.
17.—The composition or the kit of parts according to any one of clauses 2-11, or the non-viral vector according to any one of clauses 12-16, for the diagnosis of a disease or disorder.

Having generally described this invention, a further understanding can be obtained by reference to certain specific examples, which are provided herein for purposes of illustration only, and are not intended to be limiting unless otherwise specified.

### EXAMPLES OF THE INVENTION

The design of the CRISPNA systems are based on the replacement of the crRNA spacer (that redirect the Cas enzymes to their targets) by a more stable and specific PNA molecule. Since different CRISPR have different compositions, the CRISPNA design will differ depending on the origin of the CRISPR system.

### 1—CRISPNA Design for Type II Cas Applications

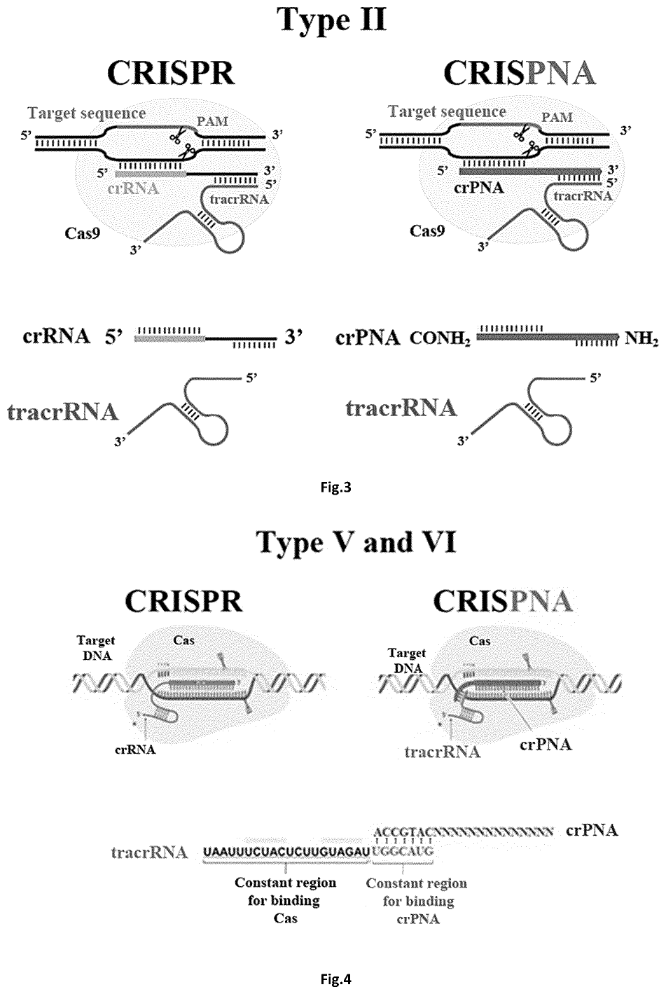

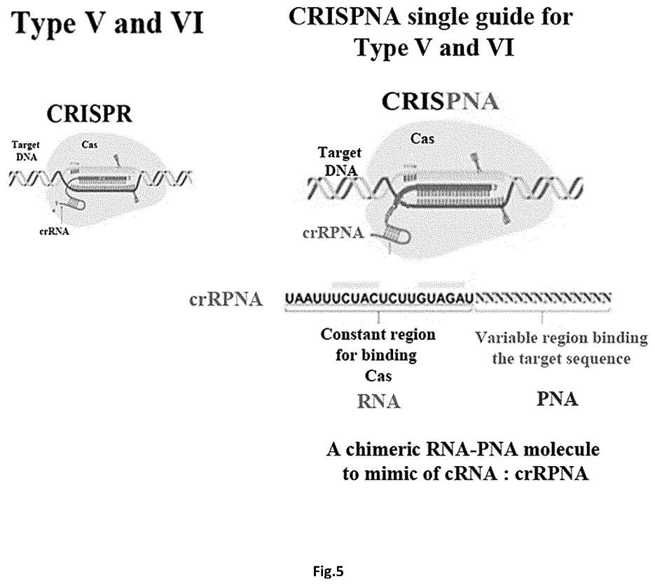

Type II CRISPNA requires the design of a PNA (named crPNA) that will replace the crRNA in the original CRISPR/Cas system while still using the tracrRNA (FIG. 3). To perform the editing/binding of Cas to their target, the crPNA, the tracrRNA and Cas protein will be mixed and added to the target samples..

### 2—CRISPNA Design for Type V/VI Cas Applications

To adapt type V and type VI CRISPR system to CRISPNA, we have two possibilities:
a) The 41-50 bp crRNA functions will be splitted into two: 1—the crTRARNA (having similar functions as the tracrRNA in type II systems) which will bind to the Cas proteins and 2—the crPNA that will direct the Cas to its target (FIG. 4).
b) To generate a chimeric molecule RNA-PNA in which the RNA domain will retain the Cas binding domain and the PNA will bind the target sequence.

### 3—Applications of CRISPNA for Genome Editing

CRISPNA/Cas is an alternative to the well-known CRISPR/Cas systems and can therefore be applied to every application of this powerful technology. One of the applications that have revolutionized basic and applied research is the possibility to manipulate DNA and RNA of living cells (genome editing). CRISPR technology has been used to develop new therapeutic strategies (Gene Therapy), to engineer stem cells, generate animal models, and to develop transgenic animals and plants that are resistant to diseases or severe conditions or have improved nutritionals values. To do so, actual CRISPR systems rely on the RNA molecules that can allow several mismatches when binding to their target leading to cut outside of the intended target (off-targets). Although for basic research this is not a mayor problem, for gene therapy applications and transgenesis is a serious concern.

PNAs display higher affinity and specificity to complementary DNA and RNA than do normal oligonucleotides and therefore, our CRISPNA system, directing the Cas proteins through PNA-DNA interactions, will be more specific and efficient than actual CRISPR systems.

### Genome Editing Examples for CRISPNA System

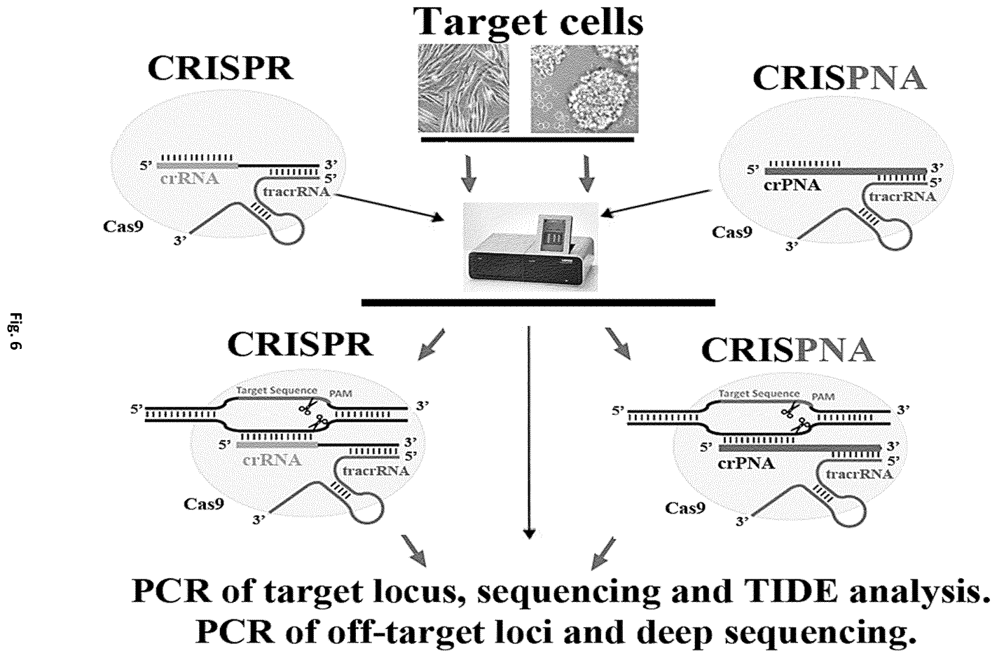

We will first generate ribonucleoprotein (RNP) complexes harboring Cas9, tracrRNA and crRNA (CRISPR) or crPNA (CRISPNA) targeting the eGFP, the GAA and the TRAC loci and, in all cases, we will perform the following common procedure (FIG. 5):

### Example 1. Genome Editing of Eukaryotic Cells

The SEWAS84S-C1 cells (Development of Cellular Models to Study Efficiency and Safety of Gene Edition by Homologous Directed Recombination Using the CRISPR/Cas9 System. Sánchez-Hernández S, Aguilar-González A, Guijarro-Albaladejo B, Maldonado-Pérez N, Ramos-Hernández I, Cortijo-Gutiérrez M, Sánchez Martín R M, Benabdellah K, Martin F. Cells. 2020 Jun. 18;9 (6):1492. doi: 10.3390/cells9061492) were used to evaluate the efficacy of genome editing in the eGFP locus. The crPNA was directed to the eGFP target, in particular to the TTGCTCACCATGGTGGCGAC sequence. To form the complex, we selected the ratio 0.5:1 (crPNA:tracPNA).

To form CRISPNA, the PNA was synthesised by Destina genomics (eGFP(N-C): TTGCTCACCATGGTGGCGAC-O-O-TCGTTTACAGATAG, where O=miniPEGspacer, 100 uM) and the chemically synthesised tracrRNA was obtained from Synthego (Silicon Valley, CA, USA) (200 μM). The crPNA-tracrRNA complex was formed at a ratio 0.5:1 (crPNA:tracrRNA) and at a concentration of 25 μM. The hybridization was performed in a thermal cycler with the following temperature reduction profile: 95° C., 5 min; 85° C., 1 min; 75° C., 1 min; 65° C., 5 min; 55° C., 1 min; 45° C., 1 min; 35° C., 5 min. Next, this crPNA-tracrRNA was mixed in a 1:2.23 ratio in terms of volume with High fidelity Cas9 (IDT, Coralville, IA, USA) and incubated at room temperature 15 min to form RNP. Then, it was delivered to cells by means of nucleofection.

### Nucleofection

Nucleofection will be performed with an AmaxaNucleofector 4-D and solution SF cell line (Lonza, Basel, Switzerland), applying program FF-120 and following the nucleofection protocol for K-562 cells. The efficiency of genome editing will be determined by TIDE analysis.

### Example 2. Generating TCRKO T Cells Using CRISPNA Technology

Primary human T cells (isolated from Apheresis products from healthy donors and activated for 48 h), will be nucleofected with CRISPR or CRISPNA RNPs designed to cut in the first exon of the constant chain of the TCRα gene (TRAC) using TCAGGGTTCTGGATATCTGT as the target sequence. To form the ribonucleoproteins (RNP) prior to nucleofection, different molar ratios will be tested, as well as several times and temperatures of incubation. T cells will be nucleofected with each RNP using P3 primary cell kit and the 4D-Electroporator (Lonza), following the protocol for stimulated human T cells (program EO-115). The efficiency of edition will be determined as described in FIG. 4 and also by flow cytometry, detecting the level of T cells that lack CD3 as a result of genome editing.

### Example 3. Measuring Homology-Directed Recombination (HDR) Efficacy in Cellular Models

We have generated different cellular model in K562 cells to evaluate the efficacy of genome editing (Sanchez-Hernandez et al under revision). Using this model and a fluorescence-based pattern we will study the efficacy of genome editing (eGFP turn-off) trigger by the CRISPR versus the CRIPNA systems. As before the crRNA and the crPNA will be directed to the same target, in this case, the TTGCTCACCATGGTGGCGAC sequence. To form the ribonucleoproteins (RNP) prior to nucleofection, different molar ratios will be tested, as well as several times and temperatures of incubation. Nucleofection will be performed with an AmaxaNucleofector 4-D and solution SF cell line (Lonza, Basel, Switzerland), applying program FF-120 and following the nucleofection protocol for K-562 cells. The efficiency of genome editing will be determined by eGFP silencing by ICE analysis (ice.synthego.com).

### Example 4. Targeting CRISPNA to SNPs

We will next explore the ability of CRISPNA to discriminate single base variations. To do this we will target an SNP present in HER2 that is associated with cardiomyopathy in patient treated with Trastuzumab. CRISPR and CRISPNA will be designed to target different SNP and the cutting efficacies of both systems will be investigated in the different haplotypes as shown before.

### Diagnostic Examples Using CRISPNA Systems

As mentioned before, in its present forms, the different CRISPR/Cas systems require RNA molecules (crRNAs or sgRNAs) to direct the different Cas proteins to their DNA or RNA targets. RNA molecules are instable and can allow several mismatches when binding to their target. Moreover, RNA hybridizations are limited to certain salt concentrations and temperatures while PNA molecules are able to hybridize complementary nucleic acid targets in a broader range of conditions.

We will develop different tools for diagnostic based on the CRISPNA system:

### Diagnostic. Example 1: Detection of KRAS Mutations

crPNA is designed to be fully complementary to the antisense strand of gDNA containing mutation G12D. crPNA is composed by a 20mers strand complementary to gDNA plus a 12mers strand which is used to hybridize tracrRNA. Cas13 is activated when mutation G12D is present, hence activating its unspecific nuclease activity. gDNA, following an amplification step is transformed to RNA using a T7 transcription step. Then, Cas13 plus crPNA and tracrRNA (Table 1) are added. In one example, a FRET reporter (reporter 1) is used. A fluorescent plate-reader is used to detect the presence of G12D mutation. In another example, a lateral flow system is used so that when a reporter is cleaved (reporter 2) it could be identified in a lateral flow system. The sequences are shown in Table 1:

TABLE 1
Sequences used to identify G12D mutations.

| Variant | DNA Sequence (5′-3′) | crPNA (N-C) |
|---|---|---|
| Wild-type | . . . AGTTGGAGCT [symbol] | GTTGGAGCTGA |
| | GGCGTAGGCAAGA . . . | GGCGTAGG [symbol] |
| | | [symbol] |
| G12D | . . . AGTTGGAGCT [symbol] | |
| | GGCGTAGGCAAGA . . . | |
| Reporter1 | 5(6)-FAM/mAmAmAmAmAm | |
| | AmA/BHQ | |
| Reporter 2 | 5(6)-FAM/mAmAmAmAm | |
| | AmAmA/biotin | |

gDNA: grey dark: codon 12; grey light: codon 13; bold letters: positions where mutations are found.
Italic: complementary region to crPNA.
crPNA: grey light: complementary region to tracrRNA molecule

### Diagnostic. Example 2: Depleting of Abundant Sequences

The use of crPNA to cleave wild-type variants before PCR amplification to provide an accurate and efficient way to enrich mutant variants of gDNA obtained from heterogeneous tumor tissues (solid and cell-free). gDNA is put in contact with preformed Cas9 plus crPNA and tracrRNA complex. The sequences are shown in Table 2:

TABLE 2
Sequences used to deplete KRAS wild type sequences

| Variant | DNA Sequence (5′-3′ ) | crPNA (N-C) |
|---|---|---|
| Wild- | . . . AGTTGGAGCT [symbol] | GTTGGAGCTGGTGGCG |
| type | GGCGTAGGCAAGA . . . | TAGG [symbol] [symbol] |

gDNA: grey dark: codon 12; grey light: codon 13; bold letters: positions where mutations are found.
Italic: complementary region to crPNA.
Underline: PAM.
crPNA: grey light: complementary region to tracrRNA molecule

### Diagnostic. Example 3: Identification of SARS-Cov2

SARS-Cov2 RNA is treated with reverse-transcriptase-recombinase polymerase amplification (RT-RPA) to amplify the S gene fragment. Then, an in vitro T7 transcription step take place before putting in contact with Cas13 complex formed by crPNA and tracrRNA (Table 3). In one example, a FRET reporter (reporter 1) is used. A fluorescent plate-reader is used to detect the SARS-Cov-2. In another example, a lateral flow system is used so that when a reporter is cleaved (reporter 2) the presence of SARS-Cov2 could be identified.

TABLE 3
Sequences used to detect SARS-Cov2.

| | | crPNA |
|---|---|---|
| Variant | Sequence (5′-3′) | (N-C) |
| S Fragment | UAACAUCACUAGGUUUCAAA | TGAAGAA |
| | CUUUACUUGCUUUACAUAGA | GAATCAC |
| | AGUUAUUUGA | CAGGAG |
| | [symbol] [symbol] | [symbol] |
| | GGUUGGACAGCUGGUGCUGCA | [symbol] |
| | GCUUAUUAUGUGGGUUAUCUU | |
| | CAACCUAGGACUUUUCUAUUA | |
| | AAAUAUAAUGAAAAUGGAACC | |
| | AUUACAGAUGCUGUAGACUGU | |
| | GC | |
| Reporter | 5(6)-FAM/mAmAmAmAmAm | |
| 1 | AmA/BHQ | |
| Reporter | 5(6)-FAM/mAmAmAmAmAm | |
| 2 | AmA/BHQ | |

RNA: grey light: area complementary to crPNA.
crPNA: grey light: complementary region to tracrRNA molecule

1. A system for recognition and cleavage of a target nucleotide, preferably a target DNA, sequence, which comprises:
(i) a Cas (clustered regularly interspaced short palindromic repeats (CRISPR)-associated proteins) polypeptide or a polynucleotide encoding a Cas polypeptide; and
(ii) a guide system comprising:
a) a scaffold RNA (tracrRNA) binding or capable of binding the Cas polypeptide of i), or a polynucleotide encoding said tracrRNA, and
b) a guide PNA (crPNA) binding or capable of binding the tracrRNA of a) and capable of binding the target nucleotide sequence.

2. The system according to claim 1, wherein the guide PNA (crPNA) consists of a structure of Formula (I) below:
Ac—NH—Y-link-Z—CONH₂ Formula (I)
wherein
Y: represents a sequence of 5-35 Peptide Nucleic Acids (PNAs) that hybridize to the target sequence;
Link: represent a 1-15, more preferably 1-10, and still more preferably 1-7 aminoethyl glycine linker between the tracRNA-binding domain and the domain that binds the target sequence; and
Z or the tracRNA-binding domain: is a PNA sequence binding the tracrRNA and having more than 5 nucleobases, more preferably 6-14 nucleobases and still more preferably about 10 nucleobases.

3. The system according to claim 1, wherein the guide PNA (crPNA) is a RNA-PNA chimera (crRPNA), consisting on the structure of Formula (II) below:
Ac—NH—Y-link-RNA Formula (II)
Y: represents the PNA guide domain, a sequence of 5-35 nucleobases that hybridizes the target sequence;
Link: represent a 1-15, more preferably 1-10, and still more preferably 1-7 aminoethyl glycine or nucleotides (of RNA) linker between the RNA and the domain that binds the target sequence; and
RNA: represents the tracRNA

4. The system according to any one of claims 1-3, wherein the Cas polypeptide belongs to the type II, type V or type VI CRISPR systems, and preferably is selected from the group consisting on: Cas1, Cas2, Cas3, Cas4, Cas5, Cas6, Cas7, Cas8, Cas9, Cas10, Cas11, Cas12, Cas13, Csy1, Csy2, Csy3, Cse1, Cse2, Csa5, Csn2, Csm2, Csm3, Csm4, Csm5 and/or Csm6.

5. The system according to any one of claims 1-4, wherein the Cas polypeptide is Cas9 and the guide system comprises:
a) A tracrRNA from any CRISPR/Cas9 system; and
b) A guide PNA (crPNA) binding the tracrRNA and capable of binding the target sequence.

6. A non-viral vector comprising the system according to any one of claims 1-5, wherein the Cas, the tracrRNA and the crPNA are mixed forming a ribonucleopeptide complex.

7. The non-viral vector according to claim 6, wherein the non-viral system is selected from the list consisting of: an electroporator, a liposome, a polycation, a nanoparticle, or combinations thereof.

8. A target cell transformed with the non-viral system of claim 6 or 7.

9. The system of any of claims 1 to 5 or the non-viral vector according to any one of claims 6 to 7, for use in therapy or medicine.

10. The system of any of claims 1 to 5 or the non-viral vector according to any one of claims 6 to 7, for the prevention, amelioration, treatment or monitoring of a disease or disorder.

11. The system of any of claims 1 to 5 or the non-viral vector according to any one of claims 6 to 7, for the diagnosis of a disease or disorder.


SEQUENCE LISTING (21 sequences)

SEQ ID NO: 1 - RNA, 67 nt, Artificial Sequence
  tracrRNA(9)
agcauagcaaguuaaaauaaggcuaguccguuaucaacuugaaaaaguggcaccgagucggugcuuu

SEQ ID NO: 2 - RNA, 15 nt, Artificial Sequence
  (II) A guide PNA
guuuaaggcuaugcu

SEQ ID NO: 3 - RNA, 21 nt, Artificial Sequence
  Formula (III)
  misc_feature 21: /note=“n= Z, wherein Z is a polynucleotide having between 5 and 70 nucleotides that hybridizes the Z sequence of the PNA sequence binding the tracrRNA of formula I”
uaauuucuacucuuguagaun

SEQ ID NO: 4 - skipped

SEQ ID NO: 5 - RNA, 35 nt, Artificial Sequence
  Formula (IV)
  misc_feature 35: /note=“n= Z, wherein Z is a polynucleotide having between 5 and 70 nucleotides that hybridizes the Z sequence of the PNA sequence binding the tracrRNA of formula I”
gauuuagaaccccaaaaacgaaggggacuaaaacn

SEQ ID NO: 6 - DNA, 36 bp, Artificial Sequence
  (eGFP(N-C)
  misc_feature 21,22: /note=“n=miniPEGspacer”
ttgctcaccatggtggcgacnntcgtttacagatag

SEQ ID NO: 7 - DNA, 20 bp, Artificial Sequence
  TCRa gene (TRAC)
tcagggttctggatatctgt

SEQ ID NO: 8 - DNA, 20 bp, Artificial Sequence
  eGFP target
ttgctcaccatggtggcgac

SEQ ID NO: 9 - DNA, 26 bp, Artificial Sequence
  Wild-type variant
agttggagctggtggcgtaggcaaga

SEQ ID NO: 10 - DNA, 26 bp, Artificial Sequence
  G12D variant
agttggagctgatggcgtaggcaaga

SEQ ID NO: 11 - DNA, 32 bp, Artificial Sequence
  crPNA table 1
gttggagctgatggcgtaggttgagattaaag

SEQ ID NO: 12 - DNA, 32 bp, Artificial Sequence
  crPNA Table 2
gttggagctggtggcgtaggttgagattaaag

SEQ ID NO: 13 - RNA, 177 nt, Artificial Sequence
  S Fragment
uaacaucacuagguuucaaacuuuacuugcuuuacauagaaguuauuugacuccuggugauucuucuucagguuggacagcuggugcugcagcuuauuauguggguuaucuucaaccuaggacuuuucuauuaaaauauaaugaaaauggaaccauuacagaugcuguagacugugc

SEQ ID NO: 14 - DNA, 32 bp, Artificial Sequence
  crPNA table 3
tgaagaagaatcaccaggagttgagattaaag

SEQ ID NO: 15 - RNA, 27 nt, Artificial Sequence
  Fig 4 tracrRNA
uaauuucuacucuuguagauuggcaug

SEQ ID NO: 16 - DNA, 21 bp, Artificial Sequence
  crPNA Fig. 4
  misc_feature 8..21: /note=“N= any nucleotide, variable crPNA that binds the chosen target sequence”
accgtacnnnnnnnnnnnnnn

SEQ ID NO: 17 - RNA, 34 nt, Artificial Sequence
  crRPNA Fig 5
  misc_feature 21..34: /note=“n=any nucleotide, variable region binding the target sequence”
uaauuucuacucuuguagaunnnnnnnnnnnnnn

SEQ ID NO: 18 - DNA, 36 bp, Artificial Sequence
  crPNA Fig 7
  misc_feature 21,22: /note=“n = MiniPEGspacer”
ttccattttcattatattttnntcgtttacagatag

SEQ ID NO: 19 - DNA, 48 bp, Artificial Sequence
  tracrRNA13 Fig 7
agcaaatgtctatccaaaatcaggggaagcaaaaaccccaagatttag

SEQ ID NO: 20 - DNA, 103 bp, Artificial Sequence
  Control sample Fig 8
gctcctcgcccttgctcaccatggtggcgaccggtggatccatcttcctgggaagggtggattatgacgggaaattctaggagaggagatgatggagagggaa

SEQ ID NO: 21 - DNA, 103 bp, Artificial Sequence; identical to SEQ ID NO: 20
  Edited sample with CRISPNA Fig 8